(19) United States
(12) Patent Application Publication
(10) Pub. No.: US 2012/0325215 A1
(43) Pub. Date: Dec. 27, 2012

(54) SELF POWERED UNIVERSAL GAS FLOW INDICATOR

(76) Inventors: William R. Levenick, Rochester, MN (US); Kathryn M. Levenick, Rochester, MN (US)

(21) Appl. No.: 13/531,438

(22) Filed: Jun. 22, 2012

Related U.S. Application Data
(60) Provisional application No. 61/500,375, filed on Jun. 23, 2011.

(51) Int. Cl. A61M 16/00

(52) U.S. Cl. 128/205.23

(57) ABSTRACT

This document provides devices and systems for monitoring the flow of breathing-gases through a gas delivery cannula. For example, in one aspect, this document provides a light, such as an LED, which is illuminated to indicate gas flow. The LED can be powered by an inline turbine or paddlewheel that is configured to harvest kinetic power from the gas and transform that kinetic energy into electrical energy.

### CROSS-REFERENCE TO RELATED APPLICATIONS

This application claims the benefit of U.S. Provisional Patent Application Ser. No. 61/500,375, filed Jun. 23, 2011.

### BACKGROUND

1. Technical Field

This document relates to methods and materials involved in monitoring gas flow (e.g., the flow of breathing-gases through a gas delivery cannula). For example, this document provides a light that can be illuminated to indicate gas flow by harvesting kinetic power from the gas with an inline energy harvesting means such as a paddlewheel or turbine.

2. Background Information

A large variety of delivery systems have been designed for administering medical grade gas to a patient. Medical grade gas is administered to a patient for various reasons and to treat various conditions. One such condition is Chronic Obstructive Pulmonary Disease (COPD). The wide range of breathing-gas devices can be exemplified by U.S. Pat. No. 4,188,946 to Watson and Rayburn.

Such breathing-gas delivery systems comprise a minimum of three elements: 1) a source of the breathing-gas; 2) a gas delivery cannula for transmitting the breathing-gas from the source to the patient, and 3) an airway interface device (AID) for introducing the breathing-gas into the patient's airway. Many breathing-gas delivery systems include additional elements incorporated into the breathing-gas source or interposed between the breathing-gas source and the gas delivery cannula. Such elements include metal conduits, “Christmas tree” connectors, wall outlets, flow meters, valves, flow regulators, and the like.

In breathing-gas delivery systems the distal end of the gas delivery cannula is connected to the breathing-gas source. The proximal end of the gas delivery cannula is continuous with or connected to the AID, which introduces the breathing-gas to the patient's airway. As long as breathing-gas is flowing through the system, the patient inhales the delivered breathing-gas as it exits the AID. If the flow of the breathing-gas through the system is cut off, the patient inhales either nothing or only ambient air, which may not have a sufficiently high oxygen content to sustain the patient. Consequently, it is important to be able to ascertain whether or not breathing-gas is flowing all the way through the gas delivery cannula to the patient's airway.

Flow-indicators can be used to determine whether breathing-gas is flowing in a breathing-gas delivery system. One type of flow indicator is a rotary sight flow indicator, which comprises a rotatable member positioned in a chamber that is in communication with the gas source. Gas moving through the chamber causes the rotatable member to rotate, and this provides a visible indication that the gas is moving. A representative general-use rotary flow indicator is disclosed by U.S. Pat. No. 4,745,877 to Chang. Such flow indicators are used in breathing-gas delivery systems, for instance, U.S. Pat. No. 6,386,196 issued to Steven Culton and also U.S. Pat. No. 7,730,847 to Redd. Additionally, flow monitors can be self-powered as in WO 2009/147,691 to Manfredi.

As noted, breathing gas is delivered to patients for various medical reasons and there is an estimated one million American's utilizing oxygen at home. Since the typical patient is elderly, and often has mobility and vision problems, and the typical oxygen delivery system utilizes a bifurcated nasal cannulae that can be tens of feet from the console (or even in a different room or different floor), there is a need in the art for an illuminated breathing-gas flow indicator located proximal to the patient which would provide additional safety, as well as peace of mind to both patients and care providers.

### SUMMARY

This document provides systems and devices for monitoring gas flow (e.g., the flow of breathing-gases through a gas delivery cannula). For example, in one aspect, this document provides a visual indicator light (e.g., an LED) that can be illuminated to indicate gas flow. The LED can be powered by an inline turbine or paddlewheel that harvests kinetic power from the gas and transforms that kinetic energy into electrical energy.

A system provided herein can include a breathing-gas source, an AID, and a gas delivery cannula for conducting breathing-gas from the source to the AID. The gas delivery cannula can have a distal end in communication with the source, and a proximal end. The flow indicator can have a housing that contains an energy harvesting device. The housing can form a chamber through which the breathing-gas flows. The housing can form at least one inlet through which the breathing-gas enters the chamber and which is in direct communication with the proximal end of the gas delivery cannula. In some cases, a housing also can form an outlet through which the breathing-gas exits the chamber and that is in communication with the AID. The flow indicator can have a rotatable member mounted within the chamber and configured to harvest kinetic energy from the gas to power a visual indicator (e.g., an LED). The rotatable member can have at least one rotatable member surface upon which the flowing gas impinges and causes the rotatable member to rotate about its axis of rotation.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a cross-sectional, side view of one example of a gas flow indicator provided herein.

FIG. 2 is a cross-sectional, top view of the gas flow indicator of FIG. 1.

FIG. 3 is a cross-sectional, side view of another example of a gas flow indicator provided herein.

FIG. 4 is a cross-sectional, side view of another example of a gas flow indicator provided herein.

FIG. 5 is a cross-sectional, side view of another example of a gas flow indicator provided herein.

FIG. 6 is a cross sectional view of another example of a gas flow indicator provided herein.

### DETAILED DESCRIPTION

This document provides systems and devices for monitoring gas flow (e.g., the flow of breathing-gases through a gas delivery cannula). For example, in one aspect, this document provides a visual indicator light (e.g., an LED) that can be illuminated to indicate gas flow. Such a visual indicator light (e.g., LED) can be powered by an inline turbine or paddlewheel configured to harvest kinetic power from the flowing gas and to transform that kinetic energy into electrical energy capable of illuminating the visual indicator light.

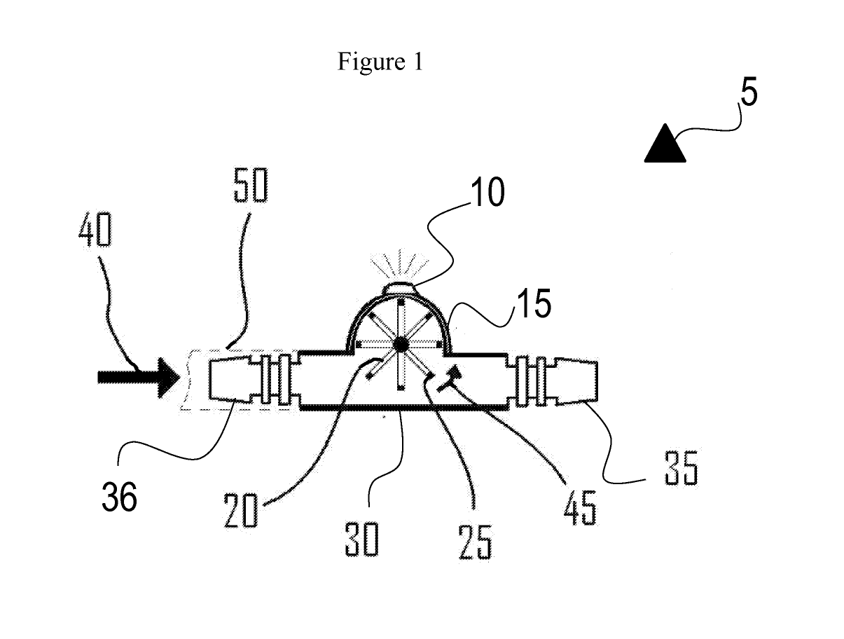

With reference to FIG. 1, a gas flow indicator 5 can include a gas kinetic energy harvesting means 20. Gas kinetic energy harvesting means 20 can be in the form of a paddlewheel or a turbine and can be located in-line with gas flow 40 (arrow 40 shows the direction of gas flow) and contained within a housing 30. In some cases, gas flow indicator 5 can include a gas kinetic energy harvestor. In some cases, gas kinetic energy harvesting means 20 can be a paddlewheel having one or more magnets 25 located at the distal end of one or more of the paddles of the paddlewheel. Magnets 25 can inductively power a circuit 15 that can be coupled to housing 30. As the paddlewheel rotates (arrow 45 shows the direction of rotation) due to gas flow 40, circuit 15 can provide power to a visual indicator 10. Visual indicator 10 can be in the form of an LED. In some cases, gas flow indicator 5 can include gas line connectors 35 and 36. Gas line connectors 35 and 36 can be used to connect gas flow indicator 5 to any suitable gas source (e.g., a breathing gas source, a portable gas source, or a welding gas source), a gas delivery cannula, an AID, or a gas supply line such as a male or female quick connect. For example, gas line connectors 36 can be used to connect gas flow indicator 5 to a gas tube 50.

A gas kinetic energy harvestor or energy harvesting means 20 may take any suitable form that has the ability to harvest energy from gas flow 40. For example, a gas kinetic energy harvestor or energy harvesting means 20 can be configured as a paddlewheel, a turbine, a radial turbine, a vertical axis turbine, a screw (such as an Archimedes' screw), a spiral (such as in a spiral pump), or a simple set of fan blades. A gas kinetic energy harvestor or energy harvesting means 20 can be in the form of any liquid based system turbine such as a water turbine or a cross flow turbine. Depending upon the type of paddlewheel or turbine used, the axis of rotation can be parallel or perpendicular to the flow of the gas without affecting the operation of the device.

In some cases, housing 30 can be shaped to maximize energy transfer from the flowing gas to an energy harvestor or energy harvesting means (e.g., a paddlewheel can be shaped like a jet engine, ramjet, or rocket booster nozzle). Such shapes can create a constrained vortex that can aid in energy transfer. In some cases, housing 30 can be formed from polycarbonate, plastic, metal, or any other form of material conducive to holding an energy harvestor or energy harvesting means in place for long-term, reliable use. In some cases, housing 30 can have a window or transparent covering to allow energy harvesting means 20 to be observed. In some cases, housing 30 can be entirely clear or made of clear plastic.

A gas kinetic energy harvestor or the energy harvesting means 20 can be configured to provide power to visual indicator 10 by use of induction, or similar appropriate techniques. As with a typical “brush-less” alternator, as the one or more magnets 25 are swept past circuit board 15, circuit board 15, having appropriate features such as stator coil windings, can have current inductively driven through them by the moving magnets. This current can then be used to illuminate visual indicator 10. Circuit board 15 can be located external to housing 30 or can be located inside housing 30 and disposed within a chamber subjected to gas flow 40, or can be integrated within housing 30. Visual indicator 10 can take the form of an energy efficient LED, a low power consumption and safe laser emitter, an LCD display, a numerical display to quantitatively indicate gas flow, luminescent organic material, light-emitting polymers, plastic scintillators, light emitting MEMS, phosphorescent organic light emitting devices, or a more traditional light bulb (e.g., an incandescent light). In some cases, visual indicator 10 can be configured in the form of an LED bar graph that can display the amount of gas flow. It will also be appreciated by those skilled in the art that multiple different visual indicators from the list above can be used as described herein. The use of small, inexpensive, light weight permanent magnets for the one or more magnets 25 can allow the device to harvest the necessary energy to power visual indicator 10, without stopping or substantially slowing gas flow.

In some cases, a gas kinetic energy harvestor or the energy harvesting means 20 in association with circuit board 15 can be configured in the form of a typical automotive alternator that relies upon rotor windings wrapped around a gas kinetic energy harvestor or harvesting means 20, which can either be held at DC, or may have a controlled magnetizing current.

With a gas flow indicator provided herein interposed between an AID and the proximal end of a gas delivery cannula, a user can easily confirm that he or she is receiving breathing-gas by visually observing an illuminated visual indicator 10, or by feeling the vibrations caused by a rotating gas kinetic energy harvestor or a rotating energy harvesting means 20. For example, a user can easily confirm that he or she is receiving breathing-gas by feeling the vibrations caused by a rotating gas kinetic energy harvestor or a rotating energy harvesting means 20. In such cases, the vibrations can be facilitated by using a counterweight or by forming an imbalance attached to the energy harvestor. In some cases, a user can easily confirm that he or she is receiving breathing-gas by observing the rotation of the kinetic energy harvestor through a transparent housing or window. The location of the gas source, which could be in the same or a different room from the user, does not interfere with the gas flow monitor's efficacy since visual indicator (e.g., LED) can be located proximal to the user. In such cases, the user can easily monitor gas flow of his or her breathing-gas through a gas delivery cannula. In some cases, a gas flow indicator provided herein can be used with portable systems.

In some cases, anyone in the vicinity of the user also can tell immediately if breathing-gas is flowing because a gas flow indicator provided herein can be positioned proximal to the user. It is not necessary to search behind drapes or other obstructions to find a flow indicator attached to the wall or to try and locate a flow indicator attached to an oxygen source that is out of sight.

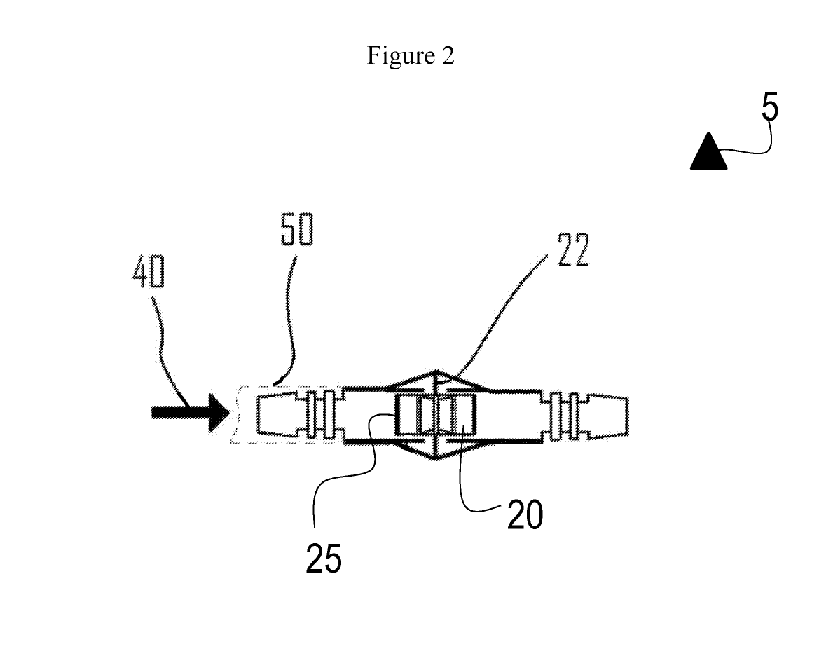

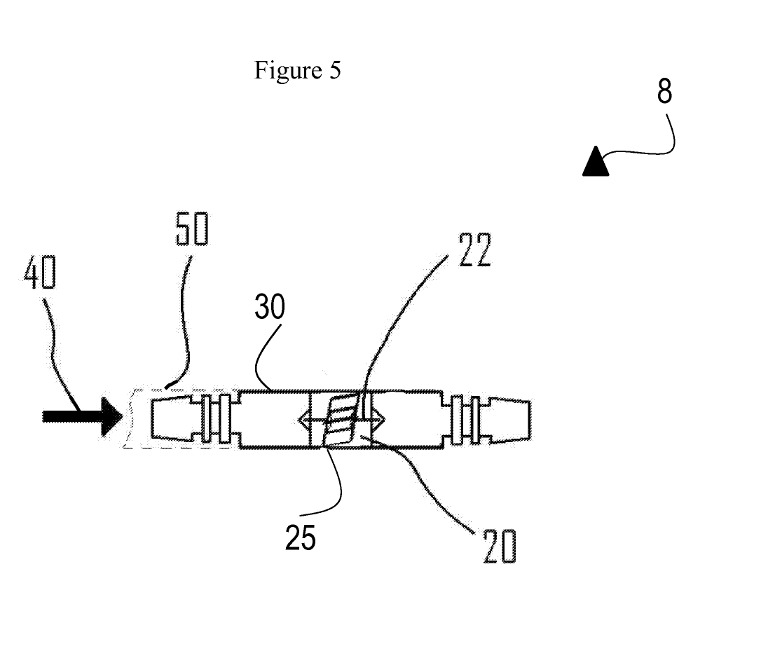

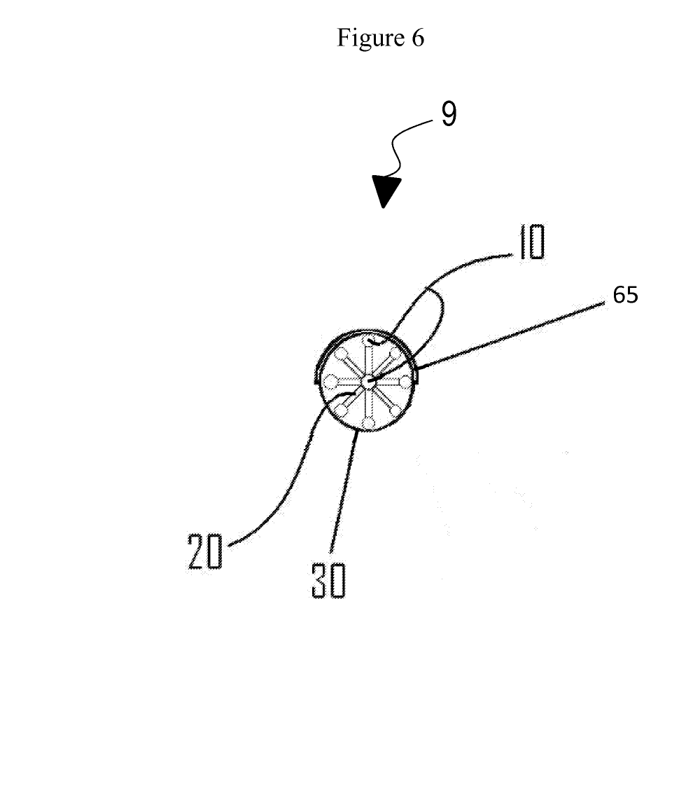

Referring to FIG. 2, a cross section as seen from above an exemplary device provided herein is presented. This view allows for visualization of an energy harvesting means axle 22. This configuration is just one example of how a gas kinetic energy harvestor or an energy harvesting means 20, which can be in the form of a paddlewheel as shown in FIGS. 1 and 2, could be mounted inside the device such that axle 22 allows for rotation of the paddlewheel around this axle due to gas flow 40. Axle 22 can be configured in any suitable form such as compression bearings and the like. Axle 22, or an alternative bearing configuration, can be made of any suitable material such as plastic, metal, ceramic, gems, and the like. If a turbine is used for harvesting means 20 rather than a paddlewheel, axle 22 can be located parallel to flow 40. An example of such a configuration is shown in FIGS. 5 and 6 as described below.

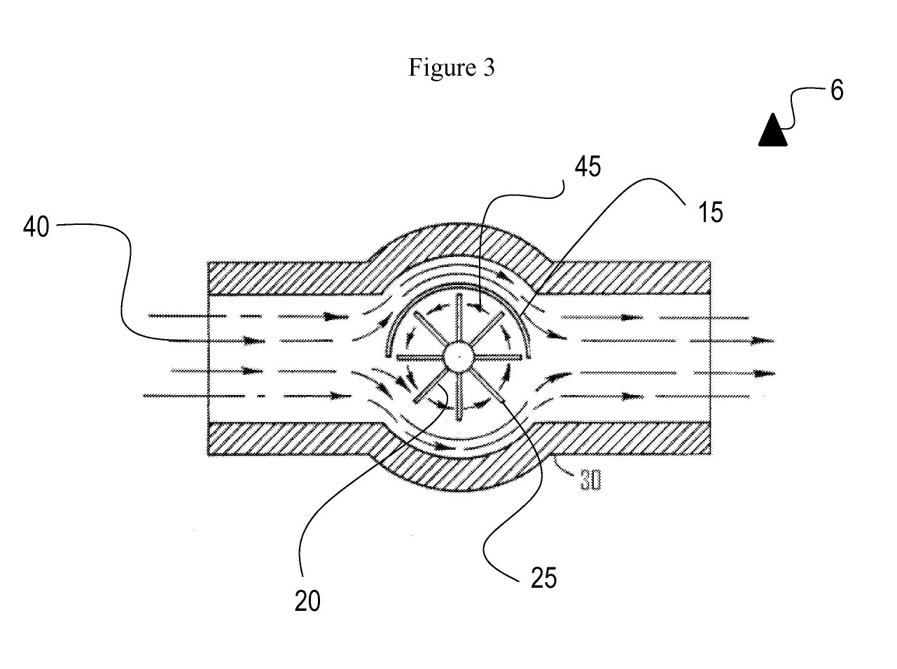

Referring to FIG. 3, gas flow indicator 6 can be configured to allow at least of portion of gas flow 40 to bypass a gas kinetic energy harvestor or an energy harvesting means 20. In this configuration, a circuit 15 can be located within a path of gas flow 40 and can be in electrical contact with a visual indicator external to a housing 30.

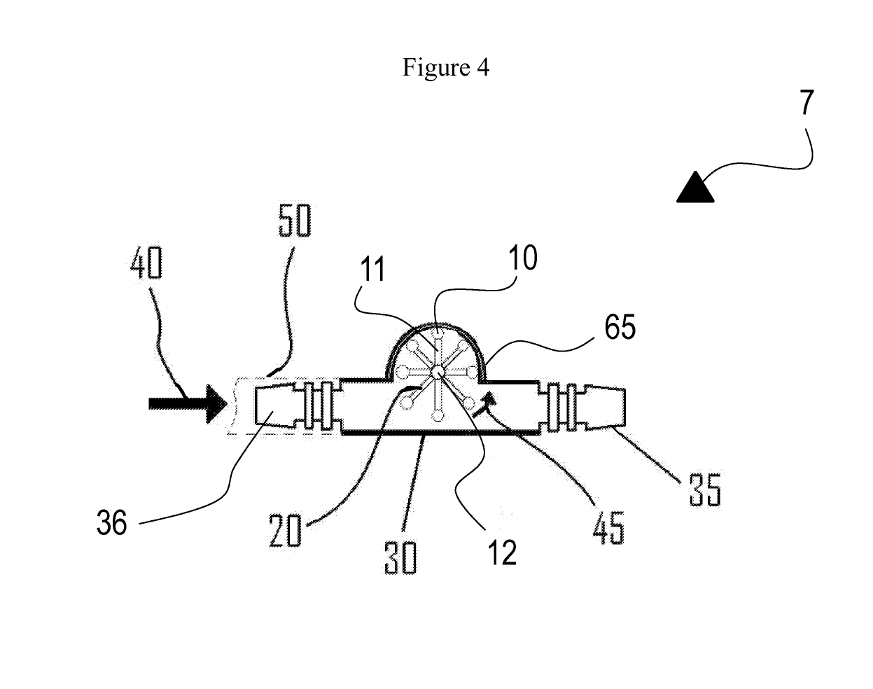

Referring to FIG. 4, a gas flow indicator 7 can be configured to include an energy harvesting means 20 that is in the form of a paddlewheel. Energy harvesting means 20 can include one or more visual indicators 10 (e.g., LEDs) that are located on one or more paddlewheel vanes 11. In some cases, one or more visual indicators coupled with coils and printed circuit boards (not shown in FIG. 4) can be located on a magnet assembly 65 (as with visual indicator 10 in FIG. 1). Magnet assembly 65 can be configured to hold one or more magnets in an appropriate configuration to inductively power visual indicators 10, which may be coupled to a circuit board (not shown, and having the necessary features, such as coils, to facilitate current induction due to relative motion with the magnets) located on or within paddlewheel vanes 11. In some cases, one or more visual indicators 12 (e.g., LEDs) can be located on an axle (the axle is not shown behind visual indicator 12 of FIG. 4). In some cases, one or more visual indicators can be located at a combination of these locations.

If one or more visual indicators are located on harvesting means 20, then the one or more magnets can be located on magnet assembly 65 or housing 30 in order to induce current. In such cases, larger magnets can be used. In some cases, each tine or vane can include a printed circuit board with an induction coil coupled to an LED. Such circuit boards can be wired together to power a common visual indicator or collection of visual indicators. In some cases, a window or an entirely transparent housing 30 can be used in order to facilitate visualization of visual indicators 10 and/or 12, which can be located within housing 30 within the path of gas flow 40.

In some configurations, a window or entirely transparent housing 30 can be used as a backup for a visual indication of gas flow, regardless of the location of the one or more visual indicators, should there be a failure anywhere in the system (e.g., a blown LED, a broken paddlewheel, and the like).

Referring to FIG. 5, a gas flow indicator 8 can be configured to include an energy harvesting means 20 that is in the form of a turbine or fan blade. An axle 22, which can be configured as described herein, can be located such that it is parallel to gas flow 40. One or more magnets 25 can be located within housing 30 or within a vane of a turbine or a blade of a fan.

Referring to FIG. 6, a gas flow indicator 9 can be configured to include one or more visual indicators 10 located on one or more vanes of a turbine, or one or more blades of a fan, or on an axle (not shown). FIG. 6 is a view is as seen from the perspective of the flowing gas (e.g., inside gas tube 50 of FIG. 1 looking in the direction of gas flow 40).

For applications where measuring gas flow in thick walled conduits is required, a paddle wheel assembly attached to a shaft can be inserted at a right angle through the wall of the conduit. Kinetic energy can be transferred via a rotating shaft with magnets or coils mounted on it as in the other described variations, with either the magnets or induction coils mounted on the housing surrounding the shaft. Current will be produced using induction to illuminate one or more visual indicators.

A device provided herein can be used in other applications including a liquid system rather than a gas environment. In some cases, a device provided herein can be used in other fields, such as on commercial airlines to provide an indicator for each passenger when the cabin oxygen supply is deployed and gas is indeed flowing through to the mask. A disposable version may be utilized as part of the bypass circuit in open heart surgeries. In some cases, the devices provided herein can be used in combination with a portable oxygen supply, a welding gas supply, or a breathing-gas supply such as an EMT type supply. Industrial applications can include providing critical flow information during a power outage, for example, as an adjunct to a pressure relief valve and/or as a feature of a male or female quick connect. A spirometer with a display could be formed with a device disclosed herein as well. Other applications include a toy that illuminates when spun on a string, home wind powered electric generation, accessory motor vehicle lights, and other ornamentation. Accordingly, it will be readily understood by those persons skilled in the art that, in view of the above detailed description of the invention, the present invention is susceptible of broad utility and application, including use with any gas flowing at anytime, anywhere along a conduit. Many adaptations of the present invention other than those herein described, as well as many variations, modifications, and equivalent arrangements will be apparent from or reasonably suggested by the present invention and the above detailed description thereof, without departing from the substance or scope of the present invention.

What is claimed is:

1. A flow indicator for indicating the flow of breathing-gas, said flow indicator comprising:
(a) a housing that defines a chamber and is configured to connect to a breathing-gas supply;
(b) an energy harvesting means located within the chamber; and
(c) a visual gas flow indicator configured to be illuminated by power generated from the energy harvesting means when breathing-gas flows past the energy harvesting means.

2. The flow indicator of claim 1, wherein the visual gas flow indicator is selected from the group consisting of an LED, an LED bar graph, an LCD display, luminescent organic material, light emitting polymers, plastic scintillators, light-emitting MEMS, phosphorescent organic light emitting devices, incandescent bulbs, and lasers.

3. The flow indicator of claim 1, wherein the energy harvesting means is selected from the group consisting of a paddlewheel, a turbine, a screw, and a set of fan blades.

4. A method of monitoring the delivery of a gas to a person's airway, said method comprising:
(a) providing a flow indicator between a proximal end of a gas delivery cannula and an inlet of an airway interface device, wherein a distal end of said gas delivery cannula is connected to a gas source, and wherein said flow indicator comprises:
(i) a housing that defines a chamber configured to receive gas from said gas source;
(ii) an energy harvesting means located within said chamber; and
(iii) a visual gas flow indicator configured to be illuminated by power generated from the energy harvesting means when gas flows past said energy harvesting means; and
(b) observing illumination of said flow indictor.

5. The method of claim 4, wherein said visual gas flow indicator is selected from the group consisting of an LED, an LED bar graph, an LCD display, luminescent organic material, light emitting polymers, plastic scintillators, light-emitting MEMS, phosphorescent organic light emitting devices, incandescent bulbs, and lasers.

6. The method of claim 4, wherein said energy harvesting means is selected from the group consisting of a paddlewheel, a turbine, a screw, and a set of fan blades.

7. A flow indicator for indicating the flow of breathing-gas within a tube from an air source to a patient, wherein said flow indicator comprises:
(a) an indicator configured to provide a visual indication to a user when air is flowing within said tube from said air source to said patient, and
(b) an energy harvestor configured to provide energy captured from air flowing within said tube to said indicator, wherein said energy is capable of powering said indicator to provide said visual indication to said user when air is flowing within said tube from said air source to said patient.

8. The flow indicator of claim 7, wherein said indicator is selected from the group consisting of an LED, an LED bar graph, an LCD display, luminescent organic material, light emitting polymers, plastic scintillators, light-emitting MEMS, phosphorescent organic light emitting devices, incandescent bulbs, and lasers.

9. The flow indicator of claim 8, wherein said energy harvestor is selected from the group consisting of a paddlewheel, a turbine, a screw, and a set of fan blades.
